SEQUENCE LISTING (6 sequences)

SEQ ID NO: 1 - DNA, 38 bp, Artificial Sequence
  Description of Artificial Sequence Synthetic primer
gccggcatatgaacaatccattggtaaatcaggctgct

SEQ ID NO: 2 - DNA, 36 bp, Artificial Sequence
  Description of Artificial Sequence Synthetic primer
tttaaaagcttttattgcacaggctgttggcgtttc

SEQ ID NO: 3 - DNA, 38 bp, Artificial Sequence
  Description of Artificial Sequence Synthetic primer
ctcagtactggatccataatcaggatcaataaaactgc

SEQ ID NO: 4 - DNA, 39 bp, Artificial Sequence
  Description of Artificial Sequence Synthetic primer
agctagtactggatcctgtattacgcactaacaggggcg

SEQ ID NO: 5 - DNA, 2154 bp, Neisseria meningitidis
  modified_base (1654): a, c, g, t, unknown or other
atgaacaatccattggtaaatcaggctgctatggtgctgccggtgtttttgttgagtgcttgtttgggcggaggcggcggcagcttcgatcttgattctgtcgataccgaagccccgcgtcccgcgccaaagtatcaagatgtttcttccgaaaaaaccgcaagcccaaaagaccaaggcggatacggttttgcaatgcgcttcaagcggcggaatcggcatcctatggtgattccgaaagaaaccgaggttaaactgaacccaaatgattgggaggcgacgggattaccgacagaacccaagaaactgccattaaaacaagaatccgtcatttcaaaagtacaagcaaacaatggcgacaacaacaatatttacacttccccctatctcacgcaatcaaaccatcaaaacggcaacactggcaacggtgtaaaccaacctaaaaatcaggcaaaaggttacgaaaatttccaatatgtttattccggctggttttacaaacatgctaaaccaaccatagatcaatcccaaaaaaaatttcaacaaggcgacgacggttatatcttttatcacggcaaagaaccttcccgacaacttcccgcttctggaaaagttacctacaaaggtgtgtggcattttgtaaccgatacgaaacagagacaaaaatttaacgatattcttgaaacctcaaaagggcaaggcgacaaatatagcggattttcgggcgatgaaggcgaaacaacttccaatagaactgattccaaccttaatgggaatcacgagggttatggttttacctcgaatttagaagtggatttcgacaataagaaattgacgggcaaactgatacgcaataatagagttacaaacgctactactggcgacaaacataccacgcaatattacagcctcgaggctcaagtaacaggcaaccgcttcaacggcaaggcgatggcaaccgacaaacccggcaacggcgaaaccaaacaacatccctttgtttccgactcgtcttctttgagcggcggctttttcggcccgaagggtgaggaattgggtttccgctttttgagcgacgataaaaaagttgcggttgtcggcagcgcgaaaaccaaagacaaagacgcaaatggcaatactgaggcggcttcaggcggcacaggtgcggcagcatcgggcggtgcggcagctatgccgtctgaaaacggtaagctgaccacggttttggatgcggtcgagctgacgcacggtggcacagcaatcaaaaaactcgacaacttcagcaacgccgcccaactggttgtcgacggcattatgattccgctcctgcccgaggcttccgaaagtgggaacaaccaagccaatcaaggtacaaacggcggaacagcctttacccgcaaatttgcccacacgccgaacagcgatgaaaaagacacccaagcaggtacggcggagaatggcaatccagccgcttcaaatacggcaggtgataccaatggcaaaacaaaaacctatgcagtcgaagtctgctgttccaacctcaattatctgaaatacgggttgctgacacgcaaaaccccggcaacacggtggaaggcggcaacggcagcccaaccgccgcccaaacggcacagggtgcaccaaagtatgttcntccaaggcgagcgcaccgatgaaaacaagattccaagcgagcaaaacgtcgtttatcgggggtcttggtacgggcatatcgccggcagcacaagctggagcggcaatgcttccaatgcaacgagtggcaacagggcggaatttactgtgaatttcgatacgaaaaaaattaacggcaagttaaccgctgaaaacaggcaggaggcagcctttaccattgagggaacgattcaggacaacggctttgaaggtacggcaaaaactgctgacttaggttttgatctcgatcaaagcaataccaccggcacgcctaaggcatattacacagatgccaaggtgaagggcggtttttacgggcctaaagccgaagagttgggcggatggtttgcctatccgggcgataaacaaacggaaaaggcaacggttacatccggcgatggaaattcagcaagcagtgcaactgtcgtattcggtgcgaaacgccaaaagcctgtgcaataa

SEQ ID NO: 6 - protein, 717 aa, Neisseria meningitidis
  MOD_RES (552): variable amino acid
MNNPLVNQAAMVLPVFLLSACLGGGGGSFDLDSVDTEAPRPAPKYQDVSSEKTASPKDQGGYGFAMRFKRRNRHPMVIPKETEVKLNPNDWEATGLPTEPKKLPLKQESVISKVQANNGDNNNIYTSPYLTQSNHQNGNTGNGVNQPKNQAKGYENFQYVYSGWFYKHAKPTIDQSQKKFQQGDDGYIFYHGKEPSRQLPASGKVTYKGVWHFVTDTKQRQKFNDILETSKGQGDKYSGFSGDEGETTSNRTDSNLNGNHEGYGFTSNLEVDFDNKKLTGKLIRNNRVTNATTGDKHTTQYYSLEAQVTGNRFNGKAMATDKPGNGETKQHPFVSDSSSLSGGFFGPKGEELGFRFLSDDKKVAVVGSAKTKDKDANGNTEAASGGTGAAASGGAAAMPSENGKLTTVLDAVELTHGGTAIKKLDNFSNAAQLVVDGIMIPLLPEASESGNNQANQGTNGGTAFTRKFAHTPNSDEKDTQAGTAENGNPAASNTAGDTNGKTKTYAVEVCCSNLNYLKYGLLTRKTPATRWKAATAAQPPPKRHRVHQSMFXQGERTDENKIPSEQNVVYRGSWYGHIAGSTSWSGNASNATSGNRAEFTVNFDTKKINGKLTAENRQEAAFTIEGTIQDNGFEGTAKTADLGFDLDQSNTTGTPKAYYTDAKVKGGFYGPKAEELGGWFAYPGDKQTEKATVTSGDGNSASSATVVFGAKRQKPVQ

(19) United States
(12) Patent Application Publication
(10) Pub. No.: US 2010/0055127 A1
(43) Pub. Date: Mar. 4, 2010

(54) Procedure For Expressing A Tbpb Protein On The Bacterial Surface Of An Attenuated Oral Live Vaccine As Prototype Of A Meningitis B Vaccine

(75) Inventor: Alejandro Venegas, Santiago (CL)

Correspondence Address: MILLEN, WHITE, ZELANO & BRANIGAN, P.C., 2200 CLARENDON BLVD., SUITE 1400, ARLINGTON, VA, 22201, US

(73) Assignee: Pontifiicia Universidad Catolica De Chile, Santiago (CL)

(21) Appl. No.: 12/513,553

(22) PCT Filed: Nov. 6, 2007
(86) PCT No.: PCT/US07/83750

§ 371 Date: May 5, 2009

(30) Foreign Application Priority Data
Nov. 6, 2006 (CL) 3000-2006

(51) Int. Cl. A61K 39/02; C12P 21/00; C07K 14/00; C07H 21/00; A61P 37/06

(52) U.S. Cl. 424/200.1; 435/69.1; 530/350; 536/23.7

(57) ABSTRACT

A procedure for obtaining the expression of a membrane antigen of a pathogen against which an live oral vaccine development is desirable on the surface of a negative Gram bacteria to which virulence is attenuated, or another bacteria or other Gram negative or positive bacteria with probiotic features which are compatible with the proposed expression system and that can be used as a live oral vaccine, wherein a plasmid is constructed and obtained based on the structure of pET family plasmids, with tbpB gene incorporated under the control of T7 promoter or another equivalent one, with the addition of a metabolic marker in the plasmid vector, previously cloned with its own promoter, inactivating, at the same time, the antibiotic resistance. In addition, recombinant microorganism such as an attenuated vaccine strain against group B meningitis with immunizing and protective properties against infection by Neisseria meningitidis.

### PREVIOUS ART

Meningitis is caused by several microorganisms that produce meninx inflammation (membranes which cover and protect the central nervous system). This illness produces brain damage with alterations ranging from imperceptible dysfunctions to severe damage and death. The incidence of Neisseria meningitidis, one of the main etiologic agents of this meningitis, is 310,000 cases of meningitis worldwide with a mortality rate higher than 11% (Diaz-Romero and Outschoorn, 1994). N. meningitidis can be classified into 13 serogroups depending on the specificity of the immunologic reactivity of the capsule, being A, B, C Y and W132 the most important. Additionally, it is classified by type depending on mutually excluding presence of outer membrane proteins (Omp2 and Omp3). There is a subtypification which depends on reactivity of monoclonal antibodies with 2 exposed regions of Omp1 (PorA). Thus, the nomenclature used is group:type:subtype, eg., B:15:P1.3. Today, there are in the market vaccines against meningococcal meningitis based on the capsular polysaccharides against groups A and C of N. meningitidis, but there is no one for group B. The main problem arises because the capsular polysaccharide of group B is not immunogenic and is similar to a component of the nervous tissue (sialic acid). Thus, as an alternative approach, vaccines based on membrane proteins present in vesicles isolated from bacteria are being developed for group B.

Today, there are several tetravalent capsular vaccines based on capsular extracts of groups A, C, Y and W135 (Gotschlich, et al, 1969). This formulation has been widely used in the WHO vaccination program (1976). This type of vaccine lacks efficacy among infections caused by N. meningitidis group C and A in toddlers under 2 years old and babies younger than 6 months (Ceesay, 1993; Costantino, 1992).

Vaccines for group B based on capsular polysaccharides have not been successful because of their insufficient immunogenicity due to the structural similarity of capsular polysaccharides of group B strains with sialic acid chains present in human glycoproteins. Today, vaccine development designs for group B are based mainly on extracts of mixtures of outer membrane proteins (OMPs). These results are discouraging and have motivated us to characterize and study certain OMPs of N. meningitidis, capable of inducing a bactericidal immunogenic response. New formulations with variable outcomes have been developed by different groups.

A mixture of OMPs from group B and polysaccharides from group C as a vaccine was assayed in the north of Chile, between 1987 and 1989, with a poor protection outcome (Zollinger et al, 1991).

At the same time, another formulation comprising purified OMPs of a Cuban strain of group B was successfully assayed in that country. Unlike the previous field assay carried out in Chile, in certain areas of Cuba, a 94% protection was obtained (Sierra et al., 1991). Amazingly, the same Cuban vaccine assayed in Brazil (1989-1990) showed a far lower protection level (De Moraes et al., 1992). These contradictory results explained by differences in responsive antigens of autochthonous strains clearly showed the importance of using local strains when designing a vaccine, due to the diversity of types and subtypes of N. meningitidis present in the area. In the past, such argument made Chilean health authorities to reject Cuban vaccine import to control a meningitis outbreak in Chile. A different formulation, based on OMPs vesicles was assayed in Norway with a discouraging 50-55% protection (Bjune et al., 1991). The latter focused our attention in the design of a vaccine against N. meningitidis group B, using native strains which show more epidemiologic relevance in Chile. Recently, a vaccine against group B, based on outer membrane vesicles and polysaccharides (MeNZB) has been tested in New Zealand but the results are still controversial.

There has been some progress in searching for protective antigens. An efficient formulation, however, easy to manufacture, distribute and store has encouraged inventors to consider TbpB protein of N. meningitidis as a suitable antigen presented in an attenuated strain of Salmonella to induce an immune response. Additionally, the use of attenuated Salmonella as a TbpB carrier in its surface may be more effective since Salmonella can act as an adjuvant, inducing a strong natural immune response. In a collection of 108 strains of N. meningitidis, two different isotypes for TbpB have been described (Robki et al., 2000). The isotype I, (1.8 kb gene) corresponds to 19.4% of the sample and isotype II (2 kb gene) to 80.6%. Anti-TbpB antisera against isotype I were not capable of killing a strain of isotype II and vice versa (Robki et al., 2000). The isolated gene described in the present invention corresponds to isotype II. Upon immunizing, rabbits with TbpB and TbpA as purified antigens, anti-TbpB antibodies were more bacteriocidal activity than those obtained with TbpA (West et al., 2001). Such data support our choice for TbpB.

A design of intranasal vaccine with attenuated Bordetella pertussis was assayed with the tbpB gene fused to the 3′ end of the hemoagglutinine gene of Bordetella pertussis. Despite the big size, the hybrid was secreted inducing a humoral response in mice (Coppens et al., 2001). Vaccines conjugated with capsular polysaccharide C in Great Britain have demonstrated effectiveness against serogroup C but not against serogroup B. It has also been evaluated a vaccine based on OMPs vesicles with PorA of serogroup B comprising 6 different subtypes for PorA (hexavalent vaccine) with optimal results in children (Martin et al., 2001). This vaccine has also been evaluated in Dutch children (toddler from 2-3 years and pre-school children up to 7 years) showing that IgG1 and IgG3 type antibodies are predominant and in a higher level in toddlers (de Kleijn et al., 2001). From genomic analysis of type B N. meningitidis, it has been found the antigen GNA-33 which corresponds to a lipoprotein homologous to the murein-transglycosylase enzyme of E. coli which acts as a mimetic antigen of loop#4 of PorA of serotype P1.2. Anti-GNA33 antibodies also showed bactericidal activity (Granoff et al., 2001). Despite the aforementioned progress, a vaccine prototype against serogroup B is not yet available on the market, not even yet considering the expression of the antigen on the surface of an attenuated Salmonella strain.

The main problem solved by the present invention is to have a vaccine against group B N. meningitidis which will be widely effective in preventing the disease in spite of the fact that Chilean strains are being used. We have done bioinformatic analyses using TbpB protein sequences and after alignment the showed high level homology along with Chilean TbpB sequences defining well conserved regions. This finding implies that several immunogenic epitopes could be shared among different strains coming from distant geographic origins. In Chile, meningitis is mainly caused by group B N. meningitidis, (i.e. strains B:15:P1.3 and B:4:NT) from which tbpB gene has been isolated. In order to assure wider protection the same antigen from other Neisseria meningitidis strains, the vaccine market can be expanded to other Latin-American countries and continents (Europe and Africa) where this kind of meningitis is a serious health problem.

Bacterial pathogens require Fe to show their virulence and availability of free Fe is restricted in mammal tissues. Fe usually travels among different mammalian tissues bound to protein carriers such as ferritin and transferrin. That is why bacteria capture Fe from transferrin after its binding to the TBP complex located on the bacterial surface. TbpB (Transferring binding protein B), as part of this complex, is located in the outer membrane of Neisseria meningitidis along with the other complex subunit, TbpA. When the complex binds human transferrin-Fe, the Fe ion is transported to the bacterial cytoplasm.

The present invention encompasses the isolation of the tbpB gene from 2 Chilean strains. N. meningitidis B:15:p1.3 and B:4:NT (the most prevalent ones). Both genes have been sequenced and have been inserted into the pET21a plasmid (an autonomous genetic element which can be introduced into bacteria and which is maintained stable inside the bacteria in order to produce TbpB protein). The presence of a protein band of 75-80 kDa in E. coli cell lysates that reacts with an antibody against TbpB has been demonstrated. There is a strong cross-reaction with TBP of both strains indicating they are homologous. After introducing the plasmid along with the tbpB gene into E. coli or S. typhimurium, TbpB protein was also located in the outer membrane of these bacteria, indicating that this protein can be accessible to the immune system of the vaccines, facilitating an adequate an selective response.

Meningococcal meningitis (caused by Neisseria meningitidis) normally affects population with a low incidence, however sporadically, some events with epidemic characteristics occur and there is a high risk of death among infected individuals as well as their potential colateral and late-effects. There are polysaccharide-based vaccines for groups A and C but we must remember that there is no efficient vaccine against group B of Neisseria meningitidis in the market yet.

Recently, Chiron Vaccines developed a vaccine for meningitis B based on an old Norwean prototype using 25 ug of deoxycholate extracted vesicles derived from strain NZ98/245 plus aluminium hydroxide (165 mg) as adjuvant per dosis. This vaccine is administrated by subcutaneous route and does not contain live bacteria or products derived from human blood or bovine subproducts or egg derivatives. However, after field trials on New Zealand, production was hold after suffering adverse comments. The parental prototype based on a Norwean strain was never released for wide spread use since low efficacy was reported. Data from New Zealand trial was not open to the health authorities of this country.

In Chile, Latin-America and other countries like Norway, United States and African countries, group B is the most prevalent and affects mainly children. Today, there is no preventive treatment against group B strains and therapeutic treatment is expensive and frequently requires hospitalization. Thus, this situation would be quite different if an efficient vaccine would be available.

Main advantages of the present invention are as follows:

a) Low cost of dose manufacturing since obtaining some liters of Salmonella culture does not require expensive reagents and equipments or huge facilities.
b) Simplicity of product handling regarding processing and formulation because it does not require expensive product purification methods since it is not administrated subcutaneously or by intramuscular route. In such cases, strict controls on purity and surveillance on pollutants, endo and pyrotoxins are required. The oral route accepts a higher levels of pollutants than other administration routes since they can travel through gastrointestinal tract and be evacuated.
c) The use of an attenuated strain obtained by partial removal of metabolic genes prevents from reverting to a virulent type. In any case, by using an attenuated Salmonella typhimurium as TbpB antigen carrier, neither meningitis nor typhoid fever will affect the vaccines and the maximum risk would be a kind of diarrhea similar to that caused by contaminated food intoxications. An attenuated strain colonizes intestinal tract for few days and then rapidly disappears from the host. Our data on vaccinated mice with attenuated strains revealed that 2 weeks after inoculation no bacteria are detected in internal organs such us spleen or liver
d) Attenuated Salmonella strain stimulates natural immune response, which is important in the initial step to control the illness and to trigger the adaptive immune response.
e) High efficiency of the manufacturing process because a few liters of vaccine strain culture produces thousands of doses. A rather conservative estimation suggests a yield of 1000 doses per liter of culture.
f) Oral route makes dose administration friendlier, especially with kids. Also, it induces a local mucus response in both intestinal and nasal epithelium where Neisseria meningitidis normally resides. In turn, other routes of administration such as subcutaneous, intramuscular or intraperitoneal do not normally induce a mucosal response.
g) Vaccine availability as an alternative to classic treatment of group B meningitis will reduce government health costs of treatments and will provides an alternative tool for prevention not currently available.

To date, there is no vaccine against group B N. meningitidis with such advantages at the market.

The vaccine based on this antigen may induce antibodies which could affect iron uptake by functional blocking receptor when it is recognized by these antibodies. In addition, the antibodies induced after mice vaccination have shown bactericidal activity which is complement dependant. Studies done by other authors also indicate that TbpB induce bactericidal antibodies (Robki et al., 1997). The procedure proposed by the invention for the development of a vaccine is based on the use of an attenuated strain of Salmonella which incorporates the plasmid with the tbpB gene and that presents the TbpB antigen on the bacterial surface. It has been demonstrated by the inventors that TbpB is actually located on the surface of S. typhimurium 4550 (the vaccine strain). Additionally, it has been found that after vaccination of BALB/c mice (pre-clinic assays), anti-TbpB antibodies were detected in blood and they showed bactericidal activity against Neisseria meningitidis in an in vitro assay. With these data, it is concluded that TbpB is a good candidate as an antigen in an attenuated oral vaccine against meningitis.

The main objective of the invention is to disclose a procedure to demonstrate that the TbpB antigen of Neisseria meningitidis can be used as a protective antigen in any attenuated vaccine system of Salmonella which expresses it on the bacterial surface. To test the vaccine in a preclinical assay a murine model was used. The requires steps are described as follows:

a) Cloning tbpB gene of Neisseria meningitidis serogroup B:4NT as an antigen capable to induce an immune response against N. meningitidis by using an attenuated Salmonella typhimurium vaccine which expresses it.
b) Molecular characterization of the tbpB gene by nucleotide sequencing and translation thereof to establish the amino acid sequence of TbpB protein.
c) Cloning of the E. coli asd gene of by PCR and testing its functionality via complementation analysis using the E. coli χ6212 mutant strain which lacks the asd gene. Because the wild type asd gene is required for the synthesis of 3 amino acids and murein (a component of the bacterial cell wall) complementation of this mutation it is absolutely necessary for bacterial viability. This is accomplished by modification of the plasmid vector to be used for expression TbpB antigen.
d). Insertion of the previously cloned tbpB gene into the pET21a vector using the NdeI and HindIII restriction sites which were incorporated into the gene during the cloning procedure. These sites are also present in the vector polylinker site facilitating the tbpB gene ligation.
e) Development and modification of a expression vector suitable for expressing tbpB gene. Modification of the pET21a plasmid by incorporating the E. coli asd gene (previously cloned) in the middle of the ampicillin resistance gene of pET21a, addition that at the same time inactivates this antibiotic resistance gene. In case that a recombination event would cause loss of the asd gene and recovery of the ampicillin resistance gene, the strain is condemned to death because the product of the asd gene is absolutely required for bacterial survival.
f) Transfer of the modified plasmid (pET21a/tbpB/asd) to the S. typhimurium χ3730 strain which is capable to methylate (but not cut) plasmid DNA following the typical methylation pattern of Salmonella, thus protecting plasmid DNA from degradation during the final transfer to the vaccine strain. Selected vaccine strain is able to degradate foreign DNA if it is not methylated according to the Salmonella methylation pattern.
g) Transfer of modified plasmid already isolated of S. typhimurium χ3730 strain to the vaccine strain S. typhimurium χ4550.
h) Assessing the humoral immune response upon vaccination BALB/c mice as the animal models.
i) Testing the bactericidal activity of sera from vaccinated mice in order to demonstrate that vaccination induces a protective effect in this model through the induction of bactericidal antibodies. Challenge studies are not feasible with this animal model.

### BRIEF DESCRIPTION OF THE DRAWINGS

FIG. 1: Illustrates a model proposed to explain the mechanism of action of the TbpB protein complex of Neisseria meningitidis which bind human transferrin for iron uptake which is necessary for tissue colonization by the pathogen. The complex which binds transferrin has 2 subunits A and B; A is tightly anchored to the outer membrane of the pathogen forming a pore to internalize iron, and subunit B, which is slightly bound to the membrane, helps to uptake transferrin via a binding domain present in its structure. It is proposed that iron would travel to the inside of this bacterium through the tbpA pore.

FIG. 2: Illustrates a scheme of tbpB gene amplification, ligation to the plasmid vector and cloning procedure of the Neisseria meningitidis, strain B:4:NT tbpB gene into the pET21a plasmid which was further modified through insertion of the E. coli K-12 asd gene of to finally be transferred to the attenuated Salmonella typhimurium strain.

FIG. 3: Illustrates a PCR amplification of tbpB gene, using TBP-1 and TBPB-1.3 primers, from chromosomal DNA of N. meningitidis B:4:NT. The PCR fragment was visualized and purified by 1% agarose gel electrophoresis. Lane St, standard 1 kb molecular-weight DNA ladder (Gibco); lane 1, negative control (mixture of PCR amplification without DNA template); lanes 2 to 5, tpbB gene amplification using PCR different assay conditions. The size of the tbpB gene band was 2.1 kb which is similar to the size described for this gene in other strains of group B N. meningitidis.

FIG. 4: Nucleotide sequence of tbpB gene (which encodes subunit B of transferrin binding protein) of Neisseria meningitidis B:4:NT strain.

FIG. 5: Amino acid sequence which corresponds to the translation of the sequence of tbpB gene of Neisseria meningitidis B:4:NT strain described in FIG. 4. The sequence was obtained using the DNASTAR program.

FIG. 6: Illustrates the detection of the asd gene by PCR, using plasmid DNAs from clones obtained after insertion of this gene in the pET21a plasmid. For the amplification of the asd gene, ASDEC1 and ASDEC2 primers and plasmid DNAs from clones with tbpB gene constructions as templates were used. Amplified fragments were separated by electrophoresis in 1% agarose gel. Lane St1=λ-Hind III DNA standard (Gibco); lane St2=100 bp ladder molecular-weight standard (Gibco); lane 1=negative control assay with no template added; lane 2 and 3=asd gene amplifications of clone 7 (construction pET-tbpB/asd), lanes 4 and 5=asd amplifications of clone 10; lanes 6 and 7 asd amplifications of clone 4, lanes 8 and 9 asd amplifications of clone 6.

FIG. 7: Scheme of the dual expression system in Salmonella typhimurium χ4550. Two plasmids are required, one for the tbpB gene expression (which carries tbpB gene ligated into pET21a, under the control of T7 promoter and inducible by IPTG) and the other one, pGP1-2, (which carries the RNA polymerase gene of phage T7 and can be induced by temperature raise from 30° C. to 42° C.).

FIG. 8: Illustrates the detection of the expression of the TbpB antigen in S. typhimurium χ3730, through the dual plasmid system. The expression was carried out after transforming this strain (which previously contained the pGP1-2 plasmid) via electroporation with the pET21a/tbpB/asd plasmid isolated from E. coli χ6212 strain. The TbpB antigen was detected in bacterial lysates induced and not induced with 1 mM IPTG at 37° C. The lysates were separated in a 12% polyacrylamide-SDS gel and TbpB was visualized after Western blot transfer according to the text. Lane c1=negative control using lysate of S. typhimurium strain χ3730; lane c2=positive control of lysate containing the construction pET21/tbpB/asd expressed in E. coli JM109(DE3) strain; lane St=wide range protein standard (Biolabs); lanes 1 to 4=lysates of 4 transformants with the pET/tbpB/_{/}asd/ and pGP1-2 dual system derived from clone 10, without induction; lanes 5 to 8=lysates of the same 4 transformants after 1 mM IPTG induction for 6 hours.

FIG. 9: Illustrates the detection of the N. meningitidis TbpB antigen expression in S. typhimurium χ4550, using the dual plasmid system. The expression was carried out after transforming this strain which previously contained pGP1-2 plasmid via electroporation with the pET21A/tbpB/asd plasmid previously isolated from S. typhimurium χ3730 strain. Bacterial lysates IPTG-induced and not induced for 6 hours at 37° C. were separated in a 12% polyacrylamide-SDS gel and TbpB was detected after Western blot transfer according to the text. Lane c1=negative control of S. typhimurium χ3730 lysate; lane c2=positive control of lysate from E. coli JM109(DE3) strain carrying the construction pET21/tbpB/_{/}asd; lane St=wide range protein standard (Winkler); lane 1=negative control (S. typhimurium χ4550) lysate; lanes 2 to 6=lysates of 4 transformants with the construction pET/tbpB_{//}asd and /pGP1-2 dual system derived from clone 10 induced with 1 mM IPTG; Lane 2=tbpB gene did not show expression.

FIG. 10: Illustrates a graphic describing serum response in vaccinated BALB/c mice with S. typhimurium χ4550 strain which carries the N. meningitidis TbpB antigen. An ELISA assay was performed (see text for details) using purified TbpB antigen bound to the ELISA plate and the graphic shows total IgG values as absorbance at 405 nm for different serum dilutions of vaccinated BALB/c mice, including one control with PBS and sera from pre-immune mice, both with primary and secondary (booster) immunization with S. typhimurium χ4550 containing both pET21-tbpB/asd and pGP1-2 plasmids.

FIG. 11: Illustrates a graphic describing IgA response in BALB/c mice feces after being vaccinated with S. typhimurium χ4550 strain carrying the TbpB antigen of N. meningitidis. An ELISA assay was performed (see text for details) using purified TbpB antigen bound to the plate, The graphic shows the values of total IgA as absorbance at 405 nm for different fecal dilutions from the vaccinated BALB/c mice, including one control with PBS and feces from pre-immune mice, both with primary and secondary (booster) immunization with S. typhimurium χ4550 containing the pET21-tbpB/asd and pGP1-2 plasmids.

FIG. 12: Illustrate a bar graphic with titers of responses of serum IgG (panel A) and fecal IgA (panel B) induced by oral immunization with the TbpB antigen of N. meningitidis expressed in S. typhimurium χ4550 vaccine strain. The response was determined through ELISA and titers reached by serum and feces samples of each group of mice were calculated. Titers were established as the highest dilution where O.D.₄₀₅ was statistically higher (p<0.05) than the values of respective pre-immune samples.

FIG. 13: Scheme of the procedure to determine bactericidal activity against N. meningitidis in BALB/C mice serum previously vaccinated with the attenuated S. typhimurium χ4550 strain containing the plasmids pET21a/tbpB/asd and pGP1.2.

### DETAILED DESCRIPTION OF THE INVENTION

The invention comprises a plasmid design and a process for construction of this plasmid expressing a protective antigen against N. meningitidis in an attenuated Salmonella strain. The plasmid design includes insertion of the tbpB gene in a pET plasmid to keep this gene under the control of the T7 promoter already present in the pET plasmid and the addition of a metabolic marker (the E. coli asd gene) to avoid the use of a plasmid with the ampicillin resistance gene (a feature not appropriate for a vaccine with potential human use). Also, this plasmid requires a second plasmid inside the vaccine strain to allow the expression of the TbpB antigen.

Process stages are as follows:

1.—Cloning of tbpB gene from Neisseria meningitidis B:4:NT by PCR amplification and gene ligation into the pGEM-T plasmid.
2.—Subcloning of tbpB gene in the pET21a expression plasmid.
3.—Expression analysis of the tbpB gene and testing best bacterial growth conditions to optimize recombinant TbpB protein synthesis.
4.—Insertion of the asd gene into pET21a/tbpB plasmid to replace the use of the antibiotic resistance marker (ampicillin) by a metabolic marker to complement chromosomal asd mutation.
5.—TbpB expression assay in an E. coli strain carrying an asd mutation.
6.—Plasmid transfer to a Salmonella typhimurium strain with a mutation in the asd gene that provides DNA methylation of plasmids constructions allowing protection to these plasmids when are finally transferred to the vaccine strain which has its restriction/methylation system in operative status
7.—Plasmid transfer to the attenuated vaccine strain carrying the mutated asd gene to be complemented by the wild type asd gene present in the modified pET21a/Tbp/asd vector.

All the above stages led to a formulation of a pre-clinic assay to evaluate humoral response (antibodies) in an oral vaccination procedure according to the protocol already established in mice. Then, the experimental approach was performed regarding whether anti-TbpB antibodies induced in vaccinated mice have bactericide activity (serum ability of killing Neisseria meningitidis in an in vitro assay).

In the following examples, each stage of the invention procedure is explained in details.

After completing the above stages a plasmid product was obtained. This plasmid derived from pET21a which comprises the tbpB gene of the Chilean Neisseria meningitidis strain B:4:NT, under the control of T7 promoter.

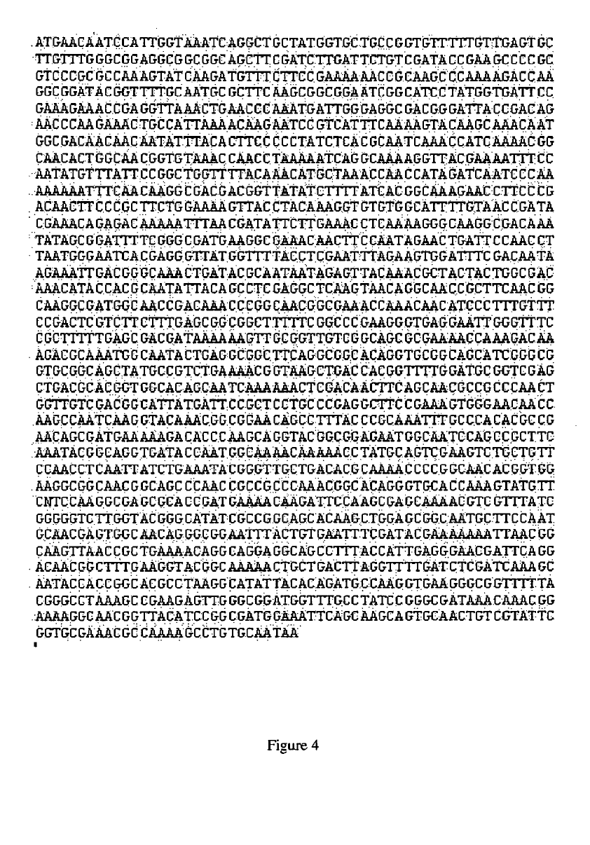

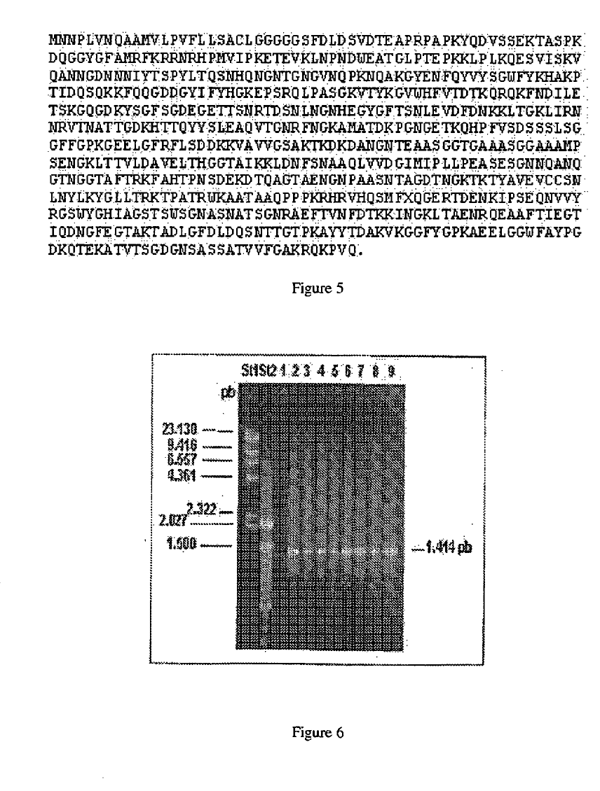

It was also possible to establish the tbpB gene sequence of the Chilean N. meningitidis B:4:NT strain in particular. The specific sequences are illustrated in the FIGS. 4 and 5.

Therefore, the oral vaccine against meningitis B is formulated in this way. To achieve this, it was required an attenuated Salmonella typhimurium strain as a vector, like the one disclosed in the provisional patent pending, 1047-2004, of the same bearer, or the Salmonella typhimurium χ4550 which was used to demonstrate the functionality of the afore mentioned plasmid.

### ILLUSTRATIVE EXAMPLES

The invention discloses a procedure to construct a plasmid which allows the expression of the Neisseria meningitidis TbpB surface antigen, which gene was cloned and sequenced. This plasmid can be used as a source to synthesize a protective antigen as part of an oral vaccine based on some type of attenuated Salmonella strain such as Salmonella typhimurium χ4550. The plasmid required to be modified by insertion of the asd gene in order to be stabilized in the Salmonella vaccine strain. At the same time, it was demonstrated that the TbpB antigen, from a Chilean strain of group B N. meningitidis, induces an IgG serum response and these antibodies showed bactericidal activity against N. meningitidis serogroup B, which confirms that TbpB antigen is a protective antigen when assayed in a murine model.

### Example 1

### Amplification of tbpB Gene from Neisseria meningitidis Group B

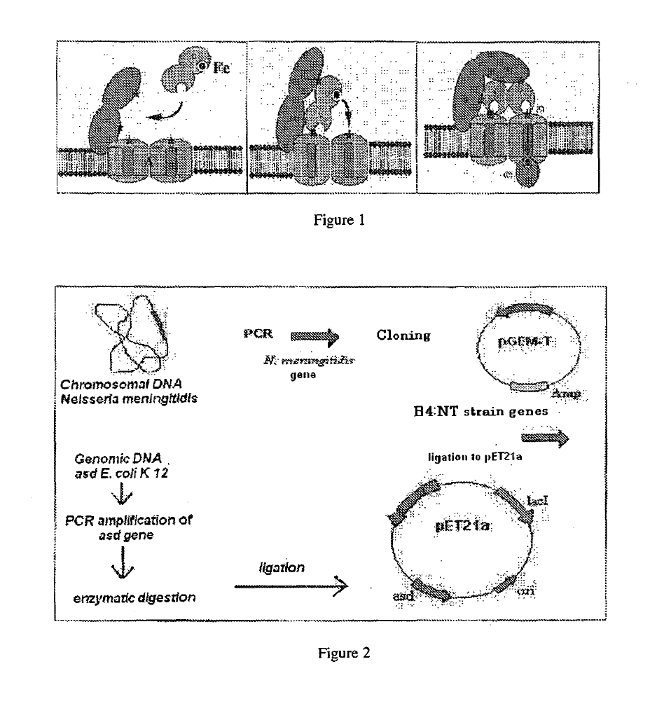

TbpB protein (B protein which binds human transferrin) is found in the surface of the Neisseria meningitidis bacteria and is part of a complex with TbpA, which is necessary for iron uptake. (See FIG. 1, scheme). Free iron is a scarce element in the host and this ion is transported through host body fluids associated to carrier proteins. Therefore, this pathogen has developed virulence factors for iron uptake from substances that transport iron in the host such as human transferrin. Several pieces of data suggest that TbpB could be a good antigen for vaccine development.

With all the above information chromosomal DNA from a N. meningitidis B:4 NT strain obtained from the Instituto de Salud Pública de Chile, purified by a standard procedure and used as a model to amplify the tbpB gene in a PCR reaction under conditions described below. The experimental strategy is illustrated in FIG. 2.

The amplification reaction was performed in a final volume of 100 μl in buffer solution of 20 mM Tris-HCl, pH 8.4; 50 mM KCl; 1.5 mM MgCl₂; 200 μM of each deoxynucleoside triphosphate (dATP, dGTP, dCTP and dTTP) and primers at a final concentration of 0.5 μM (50 pmol/100 μl). Primer sequences were as follows:

TBP-1
5′ GCCGGCATATGAACAATCCATTGGTAAATCAGGCTGCT 3′
TBP-1.1
5′ TTTAAAAGCTTTTATTGCACAGGCTGTTGGCGTTTC 3′

These primers included a Nde1 site for the 5′ end of the gene and a HindIII site (both underlined) for the 3′ end of the gene.

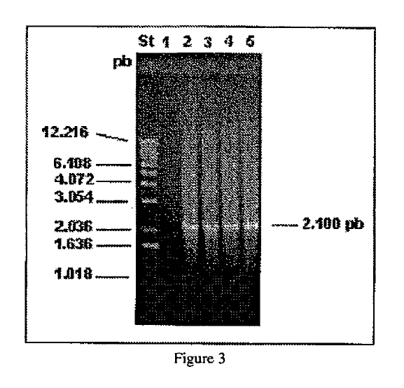

Before the amplification, the N. meningitidis genomic DNA of the respective strain (0.1-0.5 μg), already present in the reaction mixture, was denature at 95° C. for 5 min. Then, 0.5 units of Pfu DNA polymerase were added and tubes were coated with a drop of mineral oil. Alternatively, in other assays Taq DNA polymerase (0.5 U per tube) was used. Each reaction mixture was subjected to 35 cycles of amplification in a MJ Research thermocycler. Each cycle consisted of 2 min at 95° C. (denaturation step), 2 min at 52° C. for annealing with TBP-1 and TBP-1.3 primers, and 4 min at 72° C. for product elongation. Finally, samples were incubated for another 10 min at 72° C. and kept at 4° C. until analyzed. The amplified fragment was analyzed in a 1% agarose gel electrophoresis under standard conditions. The size of the fragment obtained was 2.1 kb (FIG. 3).

### 1.1.—Agarose Gel Electrophoresis of DNA.

Fragment separation between 0.5 and 10 kb was performed by electrophoresis en horizontal agarose gels (0.8-1.0%) prepared in TAE solution (40 mM Tris-HCl, 2 mM EDTA, 20 mM sodium acetate adjusted to pH 8.0 with glacial acetic acid). Gels were submitted to electrophoresis in TAE buffer, at 50 mA for analytical gels, and 30 mA for preparative gels. Before placing samples into the gel, these were incubated for 5 min a 65° C. with an equal volume of 2× sample buffer (25% glycerol, 0.5% SDS, 0.025% bromophenol blue and 12 mM EDTA). DNA bands in agarose gels were visualized on a UV transiluminator, previously stained for 10 min in a 1 μg/ml ethidium bromide (Et-Br) solution. Gels were photographed under UV light, using Polaroid type 667 films.

### Example 2

Ligation of the tbpB Insert into the pET21a Expression Vector Followed by Transfer to DH5α E. coli to Obtain Plasmid DNA Necessary for Vector Modification.

The fragment excised from the agarose gel was purified using a QIAGEN kit. The DNA fragment containing the tbpB gene was released by double digestion with NdeI and HindIII enzymes under standard conditions and ligated to the pET21a vector (Novagen) using DNA ligase and 1 mM ATP (Sambrook et al., 1989). An aliquot of the ligation reaction containing 50 ng of ligated DNA was used to transform by electroporation E. coli DH5α cells. This bacterium had the following genotype: F′ endA1 hsdR17 (rₖ⁻ mₖ⁺) gln V44 thi-1 recA1 gyrA (Nal^{r}) relA1 Δ(lacZYA-argF)U169 deoR(φ80 LacAΔM15).

### 2.1—Preparation of Competent Cells for Electroporation.

These were prepared according to the method described by Miller (1994) with the following modifications: 100 ml of bacterial culture grown in Luria medium until O.D.₆₀₀ reached values between 0.4 and 0.6, then was transferred into a sterile 150 ml Corex tube and centrifuged at 5,000 rpm in a GSA rotor (Sorvall) at 4° C. during 15 min. After discarding all culture medium, cells were maintained in ice and carefully suspended in 100 ml of sterile cold distilled water and were centrifuged again under the same conditions. Pelleted bacteria were resuspended in 2 ml of distilled cold sterile water and they were distributed into two microfuge sterile tubes. Cells were centrifuged at 5,000 rpm in the microfuge and finally, the pellet of each tube was resuspended in 100 μl of cold 10% glycerol solution. The suspension was kept frozen in 40 ul aliquots at −70° C. until used. The average yield of this preparation varied between 10⁹ and 10¹⁰ viable cells/ml.

### 2.2.—Transformation by Electroporation.

This was done according to the method described by Chasy et al., (1988), with minor modifications of Miller (1994). Bio-Rad equipment including the Gene Pulser™ power supply (version 2-89), coupled to a pulse controller was used. In a sterile microfuge tube kept on ice, 40 μl of electrocompetent cells (10⁸-10⁹ cells/ml) were mixed with 0.1 to 1.0 μg of plasmid DNA. The mixture was maintained on ice for 5 min and then transferred to a Bio-Rad electroporation sterile cuvette of 0.2 cm separation between the electrodes. The cuvette maintained on ice was then transferred into a special electroporation chamber to apply the electric pulse. The Gene Pulser™ equipment was coupled to the pulse controller unit and the electroporation conditions were fixed at 2,500 V, 200Ω resistance and 25 uF capacitance, obtaining an electric field of 12.5 kV/cm and a time constant between 4 and 5 msec. The actual electroporation conditions were verified by reading the equipment display. The DNA and cells mixture was transferred to a sterile tube with 1 ml Luria medium. The mixture was incubated at 37° C. with constant shaking during 60 min. Selection of transformants was done in 1% Luria agar plates containing 100 μg/ml ampicillin. Plates were incubated 16 to 24 h at 37° C. Many clones were obtained and some of them were sequenced in both strands using internal primers. The complete nucleotide sequence of a clone is shown in FIG. 4 and the deduced amino acid sequence obtained from it by a DNASTAR program is shown in FIG. 5.

### Example 3

### Modification of the Vector by Replacement of the Ampicillin Resistance Gene by the asd Gene (Aspartate Semialdehyde Dehydrogenase Enzyme) Used as a Metabolic Marker

In order to avoid plasmid loss by asymmetric segregation after replication in the bacterial cells, the plasmid carrying the heterologous antigen gene usually maintained within the attenuated bacteria under selective pressure using an antibiotic resistance gene included into the plasmid. Since the use of a vaccine strain with an antibiotic resistant ability is not appropriate for human use, a metabolic gene inserted into the plasmid to complement a disabled cellular function of the host as a feasible alternative for selective pressure. Therefore, to maintain the plasmid in the bacteria as long as possible, the ampicillin resistance gene in pET21a vector was replaced by the aspartate dehydrogenase gene (asd). The vaccine strain we have selected is an asd mutant that requires diaminopimelic acid (DAP) to grow. Since this metabolite is not present in mammalian extracellular fluids, insertion of the asd gene in the E. coli pET21a vector will replace the requirement of DAP for the mutant to grow. In addition, the insertion of the asd gene interrupts the ampicillin resistance gene, causing its inactivation. For this purpose it was necessary to obtain the asd gene from E. coli K-12, and to insert it into the pET21a vector containing the tbpB gene, as described in the following paragraph.

3.1.—Amplification of asd Gene from E. coli K-12.

The amplification reaction was done in 25 μl final volume of a buffer containing 20 mM Tris-HCl, pH 8.4; 50 mM KCl; 1.5 mM MgCl₂, and 200 μM of each desoxiribonucleoside triphosphate (dATP, dGTP, dCTP and dTTP), and 0.5 μM each primer (50 pmol/100 ul). Chromosomal DNA from E. coli K12 strain was isolated by the Grimberg et al. method (1989) and used as template. Primers were designed from the nucleotide sequence of the asd gene (Haziza, 1982), included promoter region and the translation stop codon, and restriction sites as indicated:

ASDEC1
5′ CTCAGTACTGGATCCATAATCAGGATCAATAAAACTGC 3′
ASDEC2
5′ AGCTAGTACTGGATCCTGTATTACGCACTAACAGGGGCG 3′

The ACTACT sequence corresponds to the recognition site of the ScaI enzyme (underlined), necessary to include the amplified fragment in the ScaI site contained in the middle of the ampicillin resistance gene in the vector. The sequence GGATCC (boldface) corresponding to a BamHI site was included as an alternative approach. The amplification and purification conditions of the asd gene were similar to that described earlier for the tbpB gene, except that Taq polymerase enzyme and the corresponding buffer provided with the polymerase were used during amplification procedure.

3.2.—Ligation of the Fragment Containing the Asd Gene from E. coli to the pGEM-T Vector and then Subcloned into the pET21a Vector.

The amplified fragment was purified and ligated to the pGEM-T vector and then used to transform DH5α cells by electroporation. Transformed bacteria were selected in Luria-agar plates containing 100 μg/ml ampicillin. The plasmid was extracted from a positive clone with the following standard procedure here described. The asd gene cloned into the pGEM-T vector was obtained by digestion with the ScaI enzyme and a 1.2 kb fragment was purified from an agarose gel with a plasmid DNA purification kit. Then, the asd gene was ligated into the pET21a vector carrying the tBPB gene and previously linearized with the ScaI enzyme. The ScaI site is located within the ampicillin resistance gene, thus this strategy allowed at the same time the inclusion of the asd gene and inactivation of the ampicillin resistance gene. The conditions for this ligation varied slightly from the ones described by Sambrook et al., (1989) since this fragment had blunt ends.

Clones containing pET21a plasmid carrying the asd gene from E. coli K12 are shown in FIG. 6.

### Example 4

### Transformation of the E. coli χ6212 asd Mutant Strain with the Modified Vector Containing the asd Wild Type Gene

After transformation of E. coli χ6212 cells with the modified plasmid vector, they become independent of DAP metabolite but, and the plasmid is stably conserved since it carries the asd wild type gene. The E. coli χ6212 strain genotype is: φ80d lacZM1 deoR Δ(lacZYA-argF)U169 supE44 λ⁻ gyrA96 (Nal^{r}) recA1 relA1 endA1, Δasd A4 Δzhf-2::Tn 10, hsdR17 (R⁻M⁺).

Thus, the pET21a/tbpB/asd prototype vector was transferred to this strain by electroporation as it has been previously described. After colony selection process in absence of DAP, clone 10 carrying plasmid pET/tbpB/asd was characterized. In order to assure that the ampicillin resistance gene was indeed inactivated and replaced by asd, parallel cultures were incubated in the presence of 100 μg/ml ampicillin. In addition clone 10 grew in plates that had no DAP, demonstrating that this clone carried a functional asd gene and also that ampicillin gene was inactive.

### Example 5

### Transfer of the pET21a/tbpB/asd Plasmid from the E. coli χ6212 Strain to the Salmonella typhimurium χ3730 Strain and Expression of the tbpB Gene in Salmonella

In order to achieve the expression of the TBPB antigen from Neisseria meningitides B:4:NT strain into the Salmonella vaccine strain it was first necessary to transfer the dual plasmid to the Salmonella typhimurium χ3730 strain, for two reasons: i).—this is a strain that has an altered system of DNA modification-restriction because a mutation in the restriction enzyme gene. It methylates foreign DNA (the required plasmids for TbpB antigen expression) but does not degrade it, facilitating the transfer of plasmids into the vaccine strain which is a wild type restriction enzyme system. A previous passage of plasmid DNA through strain χ3730 methylates this DNA avoiding degradation when transferred into the vaccine strain, since both strains share the same restriction-modification system but S. typhimurium χ3730 strain only methylates foreign DNA increasing transformation efficiency when plasmid DNA is introduced into the vaccine strain. ii) The S. typhimurium χ3730 strain is asd-mutant. Thus the incorporation of a plasmid containing a wild type asd gene ensures the independence from DAP under determined growth conditions, giving stability to the pET21a/tbpB/asd plasmid construction, since asd gene is required for bacterial survival. that has been previously constructed. The S. typhimurium χ3730 strain genotype is: leu hsdL galE trpD2 rpsL120 (Str^{r}) ΔasdA1 Δ[zhf-4::Tn10] metE551 metA22 hsdSA hsdB ilv. In addition, this strain already carried the pGP1-2 plasmid. This plasmid contains the RNA polymerase gene from T7 phage allowing the expression of the tbpB gene cloned in pET21a because is under the control of the T7 promoter.

5.1.—Transformation of S. typhimurium χ3730 Strain with pET21a/tbpB/asd Plasmid.

The S. typhimurium χ3730/pGP1-2 strain was transformed with the pET/tbpB/asd plasmid. The S. typhimurium χ3730/pGP1-2 intermediate strain is a hsd mutant with r⁻ m+phenotype, in contrast to the vaccine strain that is r⁺m⁺.

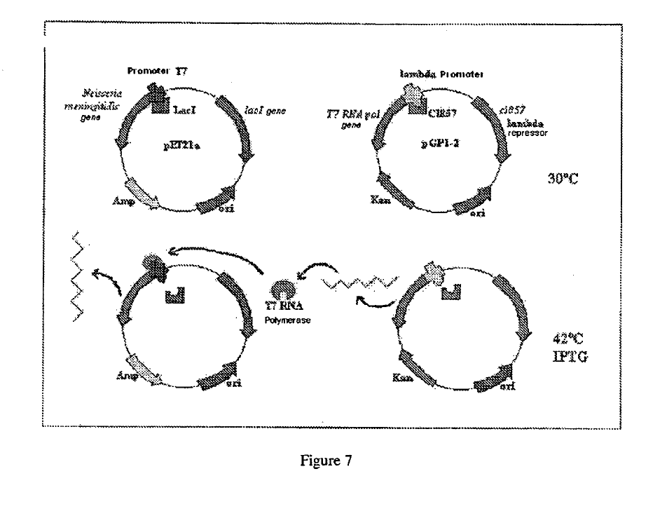

The dual system includes the pET-derivative plasmid with the gene to be expressed under the control of the T7 promoter (P_{T7}) and the pGP1-2 plasmid (Tabor and Richardson, 1985) that provides the T7 RNA polymerase (see FIG. 7). The expression is triggered by abruptly raise of the incubation temperature from 30° C. to 42° C., since the T7 RNA polymerase gene, contained in the pGP1-2 plasmid is under the left promoter of phage λ (λ P_{L}) which depends on temperature raise to function. This promoter is permanently inhibited by the temperature sensitive repressor cl857, when the temperature is under 30° C. Thus, cl857 repressor inhibits transcription at the λ P_{L} promoter at 30° C. but the repressor becomes active after the culture temperature within these cells raises briefly to 42° C. The repressor is inactivated inducing the T7 RNA polymerase gene. Thus, this enzyme promotes transcription of genes cloned in the pET vector family. Because of this, tbpB gene cloned in pET21a is under the control of P_{T7}, specifically recognized by the T7 RNA polymerase. pET21a plasmids (and its derivatives) and pGP1-2 plasmids are compatible to share the same bacterial cell and are not excluded once inside the bacterium. This is due to their different replication origins (colE1 and pA15) and also they carry different resistance markers, Amp and Kan, respectively (Ausubel, 1991), facilitating the selection pressure. Furthermore, pET21a/tBPB construction has already been modified so, instead of presenting ampicillin resistance it carries a wild type asd gene that allows the strain to growth in the absence of DAP.

Transformation has been done by electroporation as previously described and the selection in Luria medium without DAP and with kanamicyn 50 ug/ml. To verify the presence of both plasmids, 4 colonies were chose from the isolated clones, and the presence of both plasmids was verified by alkaline lysis (Bimboim and Doly, 1979) with a modification described by Sambrook et al., (1989), or alternatively, using the appropriate QIAGEN kit, following provider instructions. Two of the selected colonies had both plasmids.

5.2.—Expression Assays for E. coli(DE3) Derivatives Under IPTG Inducible Promoters.

Two milliliters of Luria broth containing the appropriate selection agent were inoculated with 0.1 ml of saturated culture containing the clones of interest and inocula were incubated with shaking at 37° C. until OD₆₀₀ reached 0.5. Then, to induce the expression of recombinant proteins, 1 mM IPTG was added to the cultures and incubated during 4 hours. Cell lysates and the Western blot analyses were done as previously mentioned.

### 5.3.—Expression Assays Using the Dual Plasmid System for Salmonella Strains.

The dual system consists of the pGP1-2 plasmid (kan^{r}), containing the T7 RNA polymerase under the control of the thermo inducible λ P_{L} phage promoter and of the plasmid derived from pET21a containing the gene of interest under the control of the T7 promoter and the asd gene inserted in this plasmid as a metabolic marker.

Inoculation of clones carrying the asd gene was done in 2 ml of Luria with 50 μg/ml kanamycin (clones with asd gene) using 50 μl (1:40 dilution) of saturated cultures grown overnight with continuous shaking at 30° C., in the case of E. coli, or at 37° C. in the case of S. typhimurium. Cultures were grown until a OD₆₀₀ of 0.4-0.6 was reached—Then, IPTG was added to a final concentration of 1 mM and cultures were kept in an incubation bath at 42° C. for 30 min to induce the expression of genes of interest under the control of T7 promoter in the pET vector. Total extracts of induced cultures were prepared and analyzed by Western blot as described below.

5.4.—Expression of TbpB Antigen in S. typhimurium χ3730 Assayed by Western Blot.

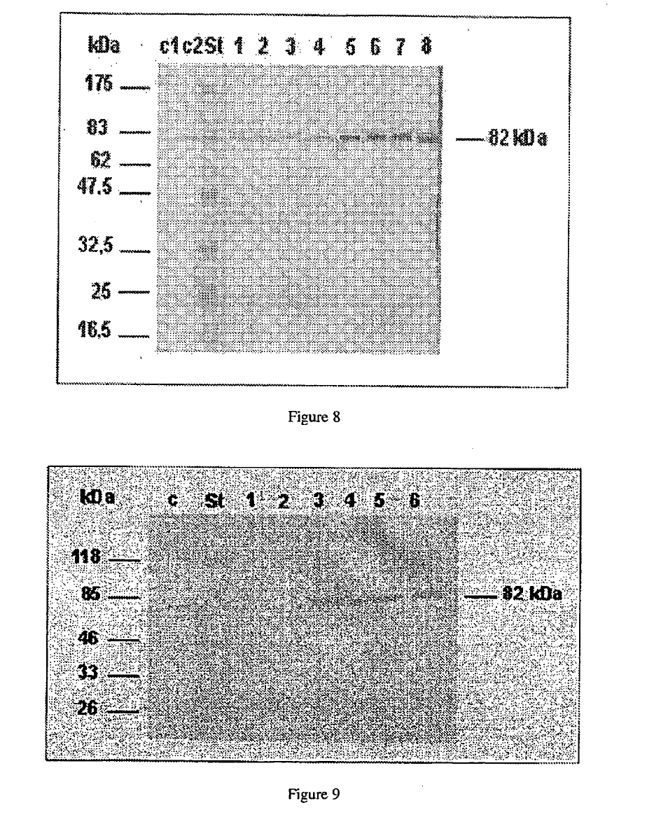

Four colonies carrying the tbpB gene were assayed. All presented a similar level of expression after induction with IPTG, as demonstrated by Western blot results (FIG. 8).

### 5.4.1.—Western Blot Assays.

This technique was done as described by Towbin et al., (1979) and it is based upon the recognition of the antigen with specific antibodies. Proteins present in bacterial lysates were previously separated by SDS-polyacrylamide gel electrophoresis (Laemmli, 1970) and transferred to nitrocellulose membranes.

### 5.4.2.—Transfer of Proteins to Nitrocellulose Filters

The unstained gel containing the separated proteins was deposited into an electrotransfer system that consists of a sponge over which is set a Whatman 3 MM filter paper, followed by the gel with the proteins, the nitrocellulose membrane, another filter paper and finally, another sponge. All this setting was supported between two perforated plastic plates. This system was submerged in a chamber containing the transfer solution (25 mM Tris-HCl, pH 8.4; 192 mM glycine and 20% methanol), carefully leaving the nitrocellulose towards the anode (+electrode) and the gel towards the cathode. The electrotransfer was carried out at 200 mA during 1 h. The nitrocellulose sheet with the electrotransferred proteins, was incubated in a blocking solution of PBS containing 1% of non-fat milk and incubated at room temperature during 45 min or at 4° C. for 10 h with continuous shakingtirring.

### 5.5—Antigen-Antibody Reaction.

After blockade of filter free sites, the nitrocellulose filter was incubated during 60 min at room temperature with rabbit polyclonal serum (1:1000 dilution) in PBS-1% milk solution. The non specific binding of antibodies was eliminated by 3 consecutive 5 min washes with washing solution (PBS-0.1% Tween 20). The specific binding of antibodies to the TrpB present in the nitrocellulose, were visualized by incubation during one hour at room temperature with an anti-rabbit IgG antibody, conjugated to horse radish peroxidase (diluted 1:1000) in the blocking solution (PBS-1% milk). After washing as previously described, the conjugate was revealed by incubation of the nitrocellulose with 50 ml of 50 mM Tris-HCl pH 7.4 containing 150 mM NaCl, to which 30 mg of 4-chloro-α-1-naphthol previously dissolved in 10 ml of cold methanol and 200 μl of 30% hydrogen peroxide were added. The reaction was stopped by extensive washing with distilled water.

### Example 6

### Plasmid Transfer from S. typhimurium χ3730 Strain to the Attenuated S. typhimurium χ4550 Strain for the Expression of the TbpB Antigen.

From the S. typhimurium χ3730 clones that expressed the TbpB antigen, four containing the pET-tbpB/asd and pGP1-2 plasmids were selected. Plasmids containing modified methylation pattern were isolated and introduced by electroporation into the S. typhimurium χ4550 vaccine strain. The selection was done by growing with 50 μg/ml kanamycin 50 ug/ml and in the absence of DAP. Grown colonies of each construction were picked up, and the above described plasmid analysis and expression studies were carried out to these transformants. Five pETtbpB/asd plus pGP1-2 containing colonies expressed TbpB. The expression of this antigen was determined by Western blot analysis as described before (FIG. 9).

### Example 7

### Mice Immunization with the Attenuated Oral Vaccine (S. typhimurium χ4550) Expressing TbpB to Evaluate the Antigen as Inducer of Protective Humoral Response Against N. meningitidis.

To determine if TbpB will be a suitable antigen to develop an oral live vaccine against the infection by N. meningitidis, the ability to induce specific antibodies after mice immunization through intra gastric pathway with the S. typhimurium χ4550 and the corresponding antigen was evaluated. For this purpose, BALB/c mice were used and divided in groups of 8 individuals, which were immunized following the steps described below.

7.1—Preparation of the Bacterial Suspension with the Vaccine Strain that Expresses TbpB.

Using a saturated inoculum, the culture was grown under continuous shaking (250 rpm) in Luria broth containing the selection agent at 37° C. until a OD₆₀₀ of 0.4 to 0.6 was obtained. Cells were recovered from an 1 ml aliquot by centrifugation and were resuspended in 4 parts of PBS and 1 part of 7.5% sodium bicarbonate in a total volume of 200 μl. The bacterial population of the suspension was estimated by extrapolating the OD₆₀₀ in a growth curve previously established, then it was adjusted to a 1×10⁷CFU that was confirmed by counting the number of viable bacteria after seeding appropriate dilutions in Luria broth agar plates.

7.2—Counting the Number of S. typhimurium χ4550 (Vaccine Strain) Viable Cells Carrying Recombinant Plasmids.

Clones of interest were grown at different stages in Luria broth with the appropriate selection agent, under shaking at 30° C. o 37° C., according to the case, until an OD₆₀₀ of 0.2, 0.4, 0.6, 0.8 and 1.0 was reached. Aliquots in each of these points in the growing culture with convenient dilutions in Luria agar plates were seeded. After overnight incubation, colonies were counted and the number of colony forming units (CFU) per ml of culture was obtained. These values were an average of at least 2 independent assays. Moreover, with these data and the time variable, a growth curve was elaborated.

### 7.3.—Oral Immunization.

Groups of 8 females, pathogen free BALB/c mice, aged 8-12 weeks were obtained from the animal facility of the Facultad de Ciencias, Pontificia Universidad Católica de Chile. These mice were immunized by oral route, applying the bacterial dose through a gastric probe of approximately 25 gauge×¾ T.W. (0.5×19 mm) diameter. The primary immunization consisted of 3 doses administered in a 6 days period, containing 1×10⁷ CFU/200 μl (OD₆₀₀=0.4) and a booster or secondary immunization as a unique dose (1×10⁷ CFU) six weeks later.

7.4.—Collection and Preparation of Samples from Immunized Mice.

One day before immunization process started (pre-immune samples) and 10 days after booster immunization, serum, saliva and feces samples were obtained. Before obtaining samples and in order to facilitate animal manipulation, mice were partially anesthetized with cotton containing a few drops of ethylic ether. Sera were obtained by centrifugation of 100 to 150 μl blood samples, collected through eye retro-orbital pathway with heparinized microcapillars (Marienfeld, Germany). Sera were kept at −70° C. until used. Feces of each mouse were collected (approximately 100 mg) and PBS with sodium azide 0.02% was added. After strong mixing in a vortex, a suspension was obtained. It was then centrifuged and the supernatant was kept under −70° C. until it was used. Antibody detection in saliva was omitted in this study because of lack of reproducibility in the collected volume, and in general, by the small amount of antibodies present in this fluid.

7.5.—Purification of TbpB from N. meningitidis by Electroelution.

To detect antibodies in mice sera and feces it was necessary to prepare a large amount of purified TbpB antigen to immobilize in the ELISA plates. Protein separation was done according to Every and Green (1982) in preparative SDS-polyacrylamide gels (15×13 ×0.3 cm), Fifteen ml samples of bacterial cultures of clones expressing TbpB in optimal conditions were used as source of protein. The electrophoresis requires 18 to 20 h at 50 V. To avoid protein staining, bands were visualized with 0.1 M KCl. The gel was cut in the band of interest, fractionated into small pieces and put into an electroelution chamber (Eluter, Bio-Rad). TbpB was obtained with a moderate yield and in reduced concentration due to its big size (approximately 80 kDa). To evaluate the obtained protein concentration, Markwell method (1978), a modification of that described by Lowry in 1951 was used. Alternatively, protein amount in aliquots of the electroeluted protein were compared to BSA dilutions of known concentration, after separation by SDS-PAGE electrophoresis. Comparison of the intensity of Coomasie blue staining obtained for different BSA concentrations allowed to estimate the approximate amount of protein obtained after electroelution.

### 7.6.—Quantitative Determination of Anti-TbpB Antibody Response by ELISA.

In order to optimize the ELISA assays, the binding efficiency of the antigen was increased by the addition of the commercial protein Pegotina™ during this process. An aliquot of 100 μl of Pegotina™ (2 ug/ml diluted in PBS, BiosChile I.G.S.A.) was added to each well from polystyrene 96 wells plates (Nunc, flat bottom). Plates were left overnight at 37° C. On the next day, plates were activated by the addition of 100 μl per well of purified TbpB (50 ng per well, diluted in PBS) and incubation at 37° C. during 2 h. Then, wells were washed 3 times, 10 min each, with PBS-0.02% Tween 20. Non-specific binding sites were blocked with 200 μl PBS-1% BSA, by 1 h incubation at room temperature. Plates were washed again with PBS-0.02% Tween 20 and dried over adsorbent towel. Double serial dilutions in PBS-1% BSA of sera and feces extract samples (100 μl) from immunized mice were added to each well. Plates were incubated for 1 hour at 37° C. and non-specific antibodies were eliminated by washing 3 times with 300 μl of PBS-0.02% Tween 20 during 5 min each time. Specific antibodies bound to the protein used to activate the solid phase were detected with mouse anti-IgG or anti-IgA antibodies conjugated to alkaline phosphatase, diluted according to vendor instructions, and incubated during 30 min at 37° C. The excess of conjugated was washed away under the same conditions previously described. Specific antibody binding was revealed after incubation with 100 μl of 1 mg/ml PNP solution (p-nitrophenylphosphate in 97 mM diethanolamine buffer with 3 mM sodium azide, pH 9.8) during 30 min at room temperature and in a dark room. Finally, the reaction was stopped with 50 μl of 3M NaOH and the hydrolyzed PNP was determined by absorbance at 405 nm in an ELISA reader (Labsystems Uniskan® I, Flow Laboratories).

7.7.—Evaluation of Data from Antibody Levels in Vaccinated Mice.

A total of two groups of mice were inoculated through the intra gastric via (one with the TbpB antigen and one control group) twice (primary immunization and secondary or booster after 50 days approximately), with 200 ul of bacterial suspension containing 1×10⁷CFU.

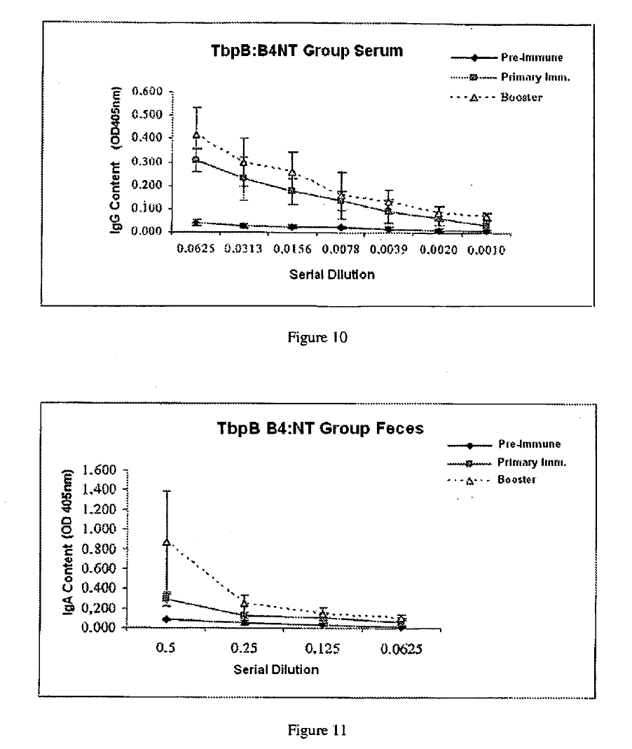

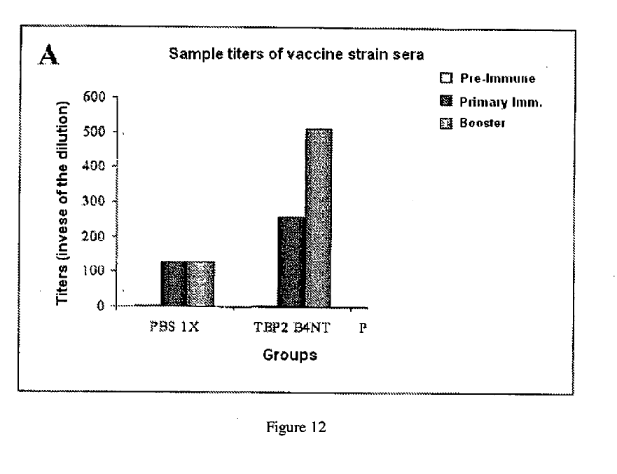

To determine if these antigens induced serum IgG and IgA antibodies in mucosal secretions (feces), blood and feces samples were taken, one month after the primary immunization and 10 days after the booster. Also, samples were taken one day before primary immunization (pre-immune samples). ELISA assays for anti-TbpB antibody determination, of IgG and IgA class, were analyzed in two forms. In one form, average ELISA values from pre-immune samples were compared to that obtained after primary and secondary immunizations with the vaccine strain expressing TbpB antigen in the serum (FIG. 10) and in feces (FIG. 11). On the other form, antibody titers reached in serum and feces were calculated, determining the highest dilution after which a statistical significant value, higher than that from the corresponding pre-immune samples was obtained (t Student test, p<0.05), for oral immunization with this antigen (FIG. 12). Moreover, the antibody response against a total S. typhimurium χ4550 vaccine strain lysate was evaluated. Values were obtained measuring OD₄₀₅. From the analysis of sample values (OD₄₀₅) obtained one month after primary immunization, 10 days after secondary immunization, and that of pre-immune samples, it was observed that group 2 of mice (pET-tpbB/as and pGP1-2 constructs, with a dose of 1.0×10⁷ cfu/100 μl) had a high antibody response with a 512 titer, statistically significant (p<0.05) compared to pre-immune serum (FIG. 12). This result was not observed in the PBS control group (no significant difference was found between OD₄₀₅ from the sample obtained after the primary immunization and that from the pre-immune sample). These observations strongly suggest that in this group intestinal colonization occurred by the vaccine strain and, therefore an appropriate stimulation with the TbpB antigen was expected.

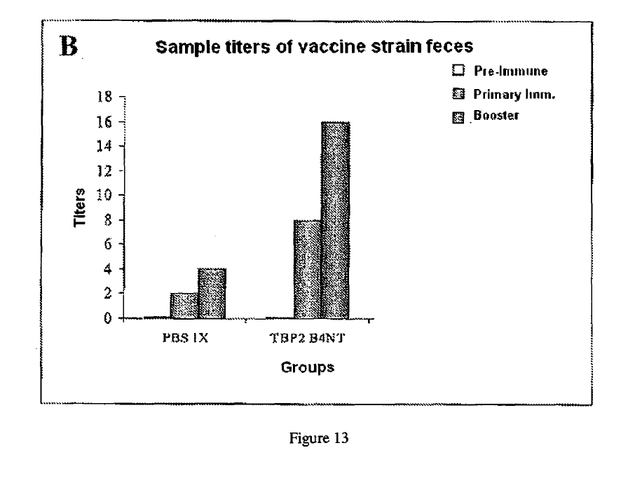

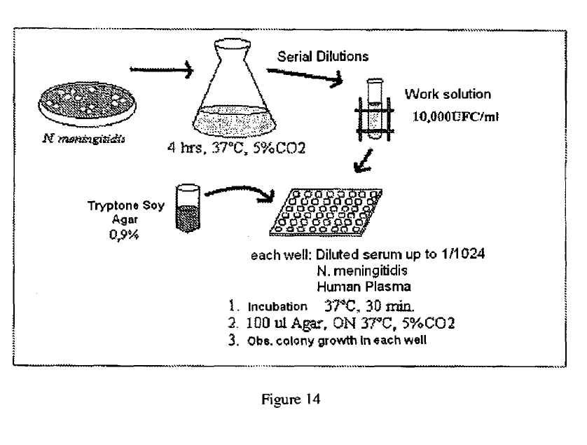

Fecal samples from mice groups immunized with TbpB presented OD₄₀₅ₙₘ values higher that those of the respective PBS control group. Fecal anti-TbpB IgA titer obtained after the secondary response was 16 (FIG. 13) in mice that received a booster 30 days after the primary immunization.

### Example 8

### Bactericidal Assays of Anti-TbpB Antibodies in Serum from Immunized Mice

To infer the protective effect of anti-TbpB antibodies produced after the immunization process, it was proposed to measure the bactericidal activity of anti-TbpB IgGs. For this purpose, we followed the procedure described by Robki et al., (1997) with modifications of a protocol for the assay of the bactericidal activity of the National Center for Infectious Diseases, C.D.C., Atlanta, Ga., U.S.A. A scheme of this assay is shown in FIG. 14.

As the pathogen for this assay, Neisseria meningitidis B4::NT strain from year 1993 (ISP strain) was used. An aliquot of N. meningitidis stored in glycerol at −70° C. was taken, and successive passages of them into agar brain-heart plates with incubations at 37° C. in 5% CO₂ (approximately 3 days) to obtain a confluent bacterial growth were made. Then, one colony was diluted into 4 ml of Hanks solution (4 mM NaHCO₃, 0.5% glucose, 0.1% BSA fraction V), pH 7.2, until a meningococcal suspension was obtained and adjusted to 1×10⁵ CFU/ml to which 2.5 U/ml heparin were added (1:50 dilution). Twenty five microliters of Hanks solution, 25 μl of immunized mice serum (primary immunization and booster diluted until 1:1024), 10 μl of pathogenic bacteria adjusted to 1×10⁵ CFU/ml, and 15 μl of normal human plasma as a source of complement were added to each well of a sterile microtiter plate.

Controls used were: bacteria viability control (40 μl of Hanks buffer and 10 μl of same bacteria dilution), complement control (25 μl of Hanks buffer plus 10 μl of bacteria and 15 μl of plasma), pre-immune mice serum control (25 μl serum plus 10 μl of bacteria and 15 μl of plasma).

After the addition of all components, the mixture was incubated at 37° C. during 30 min, time after which 100 μl of soy-tryptone agar (0.9%) was added avoided bubble formation. It was left overnight at 37° C. in 5% CO₂. Bactericidal activity was determined by counting colonies in serial dilutions with the aid of a microscope.

For this assay immunized mice sera were used and the results obtained (Table I) indicated that serum from mouse 24 contained anti-TbpB antibodies with 79.8% bactericide efficiency in the 1/32 dilution from booster serum, therefore its bactericidal calculated titer was 32.

TABLE 1
Values obtained from bactericidal assays of sera from immunized
mouse with the vaccine strain expressing tbpB gene in S. typhimurium
χ4550 tested on N. meningitidis (B4:NT serogroup/serotype).
Bactericidal activity of serum dilution

| Dilution | ½ | ¼ | ⅛ | 1/16 | 1/32 | 1/64 | 1/128 | 1/256 | 1/512 | 1/1024 |
|---|---|---|---|---|---|---|---|---|---|---|
| Primary immunization | 23 | 88 | 115 | 180 | 228 | 380 | NQ | NQ | NQ | NQ |
| Booster | 15 | 43 | 108 | 143 | 194 | 256 | NQ | NQ | NQ | NQ |

Controls with N. meningitidis B4:NT:
C₁ = NQ (non quantifiable >900 colonies)
C₂ = 964 bacteria
C₃ = NQ
Symbols:
C₁ = Complement control, 25 μl Hanks buffer plus 10 μl of bacteria and 15 μl of plasma.
C₂ = Bacterial viability control, 40 μl Hanks buffer and 10 μl of bacteria.
C₃ = No immunization serum control, 25 μl of pre-immune serum plus 10 μl of bacteria and 15 μl of plasma.

### REFERENCES

Bjune G, Gronnesby J K, Hoiby E A, Closs O, Nokleby H. (1991). Results of an efficacy trial with an outer membrane vesicle vaccine against systemic serogroup B meningococcal disease in Norway. NIPH Ann. 14: 125-132.
Ceesay S J, Allen S J, Menon A, Todd J E, Cham K, Carlone G M, Turner S H, Gheesling L L, DeWitt W, Plikaytis B D, et al. (1993). Decline in meningococcal antibody levels in African children 5 years after vaccination and the lack of an effect of booster immunization. J Infect Dis. 167:1212-1216.
Constantino P, Viti S, Podda A, Velmonte M A, Nencioni L, Rappuoil R. (1992). Development and phase 1 clinical testing or a conjugate vaccine against meningococcus A and C. Vaccine. 10: 691-698.
Coppens I, Alonso S, Antoine R, Jacob-Dubuisson F, Reauld-Mongenie G. Jacobs E, Locht C. (2001). Production or Neisseria meningitidis Transferrin-binding protein B by recombinant Bordetella pertussis. Infect. Immun. 69:5440-5446.
Curtiss III R, Goldschmidt R M, Fletchall N B, Kelly S M. (1988). Avirulent. Salmonella Typhimurium Δcya Δ crp oral vaccine strains expressing a streptococcal colonization and virulence antigen Vaccine 6: 155-160.
De Kleijn E, van Eijndhoven l, Vermont C, Kuipers B, van Dijken H, Rumke H, de Groot R, van Alphen L, van denn Dobbelsteen G. (2001). Serum bactericidal Activity and isotype distribution of antibodies in toddlers and schoolchildren after vaccination with RIVM hexavalent porA vesicle vaccine. Vaccine 20: 352-358.
de Moraes J C, Perkins B A, Camargo M C, Hidalgo N T, Barbosa H A, Sacchi C T, Landgraf I M, Gattas V L, Vasconcelos Hde G, et al. (1992). Protective efficacy of A serogroup B meningococcal vaccine in Sao Paulo, Brazil. Lancet. 340:1074-1078. Erratum in: Lancet 1992 December 19-26; 340(8834-8835):1554. Gral I M. (corrected to Landgraf I M.
Diaz-Romero J. and Outschoorn Y. (1994). Current status of meningococcal group B vaccine candidates: capsular or non capsular? Clin. Microbiol. Rev. 7:559-575.
Gotschlich E. C., Goldschneider I. Artenstein M. S. (1969). Human immunity to the meningococcus. IV. Immunogenicity of group A and group C meningococcal polysaccharide in humans volunteers. J. Exp. Med. 129: 1367-1384.
Granoff D M, Moe G R, Guiliani M M, Adu-Bobie J, Santini L Brunelli B Piccinetti F, Zuno-Mitchell P, Lee s s, Neri p, Bracci L, Lozzi L, Rapppuoil R. (2001). A Novel mimetic antigen eliciting protective antibody to Neisseria meningitidis. J. Immunol. 167:6487-6496.
Grimberg J, Maguire S, Belluscio L. (1989). A simple method for the preparation of plasmid and chromosomal E. coli DNA. Nucl. Acids Res. 17.8893
Martin S, Sadler F, Borrow R, Dawson M, Fox A Cartwright K. (2001). IgG antibody subclass responses determined by immunoblot in infants' sera following vaccination with a meningococcal recombinant hexavalent PorA OMV vaccine. Vaccine. 19:4404-4408.
Robki B, Mignon M, Maitre-Wilmotte G, Lissolo L, Danve B, Caugant D A Quentin_Millet M J. (1997). Evaluation or recombinant transferring-binding protein B variant from Nesseria mengiditidis for their ability to induce cross-reactive And bactericidal antibodies against a genetically diverse collection of serogroup B strains. Infect. Immun. 65: 55-63.
Robki B, Renault-Mogenie G, Miignon M, Danve B, Poncet D, Chabanel C, Caugant D A, Quentin-Millet M J. (2000). Allelic diversity of the 2 transferrin binding protein B gene isotypes among a collection of Neisseria meningitidis strains representative of serogroup B disease: implication for the composition of a recombinant TbpB-bases vaccine. Infect. Immun. 68: 4938-4947
Sierra G V, Campa H C, Varcarel N M, Garcia I l, Izquierdo P L, Sotolongo P F, Casanueva G V, Rico C O, Rodríguez C R, Terry M H. (1991). Vaccine against Group B Neisseria meningitidis: protection trial and mass vaccination results in Cuba. NIHP Ann. 14:195-207; discussion 208-10.
West D, Reddin K, Matheson M, Health R, Funnell S, Hudson M, Robinson A, Gorringe A. (2001). Recombinant Neisseria meningitidis transferring binding protein A protects against experimental meningococcal infection. Infect. Immun. 69: 1561-1567.
Zollinger W D, Boslego J, Moran E, Garcia J, Cruz C, Ruiz S, Brandt B, Martinez M, Arthur J, Underwood P, et al (1991). Meningococcal serogroup B vaccine protection trial and follow-up Studies in Chile. The Chilean National Committee for Meningococcal Disease. NIHP Ann. 14:211-212; discussion 213.

1-12. (canceled)

13. A procedure to obtain the expression of TbpB protein (or another membrane antigen of a pathogen against which a live oral vaccine development is desirable) on the surface of a negative Gram bacteria like Salmonella which virulence is attenuated, or another bacteria which belong to another genus such as Shigella, Bordetella, Brucella or other Gram negative or Gram positive bacteria with probiotic features which are compatible with the proposed expression system and that can be used as a live oral vaccine to express the TbpB antigen of N. meningitidis or part of its sequence comprising the following stages:
a) Cloning of the tbpB gene into a pET plasmid vector by insertion of the PCR amplified gene into the NdeI and HindIII restriction sites of the plasmid
b) Modification of the plasmid by insertion of the E. coli previously cloned asd gene into the ScaI site of the vector ampicillin resistant gene
c) Transfer of the modified plasmid to an E. coli asd mutant and then to a Salmonella asd mutant strain which carries the pGP1-2 gene
d) Analysis of TbpB expression in a Salmonella asd-strain
e) Set up for the best conditions for TbpB expression (heat pulse at 42° C. and IPTG)

14. A procedure to obtain the expression of TbpB protein (or another membrane antigen of a pathogen against which a live oral vaccine development is desirable) on the surface of a negative Gram bacteria like Salmonella which virulence is attenuated, or another bacteria which belong to another genus such as Shigella, Bordetella, Brucella or other Gram negative or Gram positive bacteria with probiotic features which are compatible with the proposed expression system and that can be used as a live oral vaccine to express the TbpB antigen of N. meningitidis or part of its sequence according to claim 13 optionally comprising the following stage:
f) Oral immunization in mice and detection of TbpB-specific antibodies with protective properties

15. A procedure to obtain the expression of TbpB protein (or another membrane antigen of a pathogen against which a live oral vaccine development is desirable) wherein a cloning stage by ligation into the pET21a plasmid according to claim 13 by the introduction of the TbpB gene obtained by PCR from Chilean Neisseria meningitidis strains B:4NT or any other nucleotide sequence with 80% homology or higher than the one described here, which were previously modified at their ends by the addition of the NdeI and HindIII restriction sites to allow the insertion of these genes at the corresponding sites on the plasmid vector.

16. A procedure to obtain the expression of TbpB protein (or another membrane antigen of a pathogen against which a live oral vaccine development is desirable) wherein the procedure requires a dual plasmid system for expression of TbpB according to claim 13, based on the structure of pET family plasmids, (pET21a) with the tbpB gene ligated into this plasmid to be under the control of the T7 promoter, and the addition of the second plasmid, pGP1-2, to the same bacterial cells in order to provide the T7 phage RNA polymerase naturally not encoded in Salmonella chromosomal DNA neither other bacterial genomes but required for efficient transcription of the tbpB gene from the T7 promoter.

17. A procedure to obtain the expression of TbpB protein (or another membrane antigen of a pathogen against which a live oral vaccine development is desirable) wherein further modification stage of plasmid carrying the tbpB gene according to claim 13 by the addition of a metabolic marker in the plasmid vector, preferably but not exclusively, the asd gene of Escherichia coli K-12 (which encodes the enzyme aspartate semialdehyde dehydrogenase), previously cloned by us with its own promoter, inactivating, at the same time, the antibiotic resistance gene, by insertion at the ScaI restriction site contained in the antibiotic resistant gene carried by the pET plasmid vector.

18. A procedure to obtain the expression of TbpB protein (or another membrane antigen of a pathogen against which a live oral vaccine development is desirable) according to claim 17 for the expression of TbpB protein, wherein both plasmids are transferred simultaneously or sequentially by electroporation into an E. coli asd-strain and then to a Salmonella asd-strain, followed by a selection in an appropriate medium and the tbpB gene is induced by IPTG and transcribed by the T7 RNA polymerase encoded in plasmid pGP1-2 which in turn is induced by raising growth temperature up to 42° C. for few minutes, being the T7 RNA polymerase necessary for the expression of TbpB (or another antigen located at the outer membrane) present in the pET plasmid which is under the control of the T7 promoter inducible by IPTG or lactose, forming a cascade effect to finally transcribe the tbpB gene and accordingly, to express the TbpB antigen detectable by Western blot in the attenuated bacteria.

19. A procedure to obtain the expression of TbpB protein (or another membrane antigen of a pathogen against which a live oral vaccine development is desirable) according to claim 18, wherein the plasmid pET-tbpB so modified is preferably adapted for expression regulated from outside (with lactose or IPTG) or inside the immunized host (by the body temperature existing inside the mouse intestine) after oral immunization using these attenuated vaccine strains.

20. A procedure stage for immunization using the TbpB protein or another antigen according to claim 19, wherein from such action, recombinant stable microorganisms such as Salmonella (which acts as a live immunizing adjuvant), are obtained as attenuated vaccine strains against group B meningitis, with immunizing and protective properties due to the ability of expressing TbpB or a part thereof on the bacterial surface and to the capability to induce bactericidal antibodies, as tested in mice.

21. Gram positive and Gram negative recombinant microorganisms such as attenuated vaccines against group B meningitis with immunizing and protective properties against infection caused by Neisseria meningitidis, obtained according to the procedure described in claim 20, wherein the ability of these modified microorganisms to express TbpB or a part thereof on the bacterial surface resides on the capability of this protein to remain bound to the host outer membrane by using an expression system wherein a cloning stage by ligation into the pET21a plasmid by the introduction of the TbpB gene obtained by PCR from Chilean Neisseria meningitidis strains B: 4NT or any other nucleotide sequence with 80% homology or higher than the one described here, which were previously modified at their ends by the addition of the NdeI and HindIII restriction sites to allow the insertion of these genes at the corresponding sites on the plasmid vector.

22. A TbpB protein obtained by the process according to claim 18, produced either in E. coli, Salmonella or other Gram negative cells, wherein such TbpB obtained from the expression of the group B Neisseria meningitidis corresponding gene and the ones obtained from their homologues, share at least 80% homology at the amino acid sequence and can be used either for immunization purposes or vaccine or for diagnosis implying the use of these antigens in tests such as ELISA, ELISPOT or immunoreactive bands which have this antigen or a part thereof included in an indicator support system which can be paper, plastic or another solid carrier where the antigen or a part of it can be chemically anchored, absorbed, or cross-linked and used for an immune reaction.

23. A TbpB antigen of Chilean Neisseria meningitidis B:4:NT strain and its gene or a part thereof from any other Chilean strain or another Neisseria meningitidis strain from other country which has an amino acid sequence with at least 80% homology or more wherein the nucleotide and amino acid sequences described as ID SEQ 1 (FIG. 4) and ID SEQ 2 (FIG. 5) respectively, as well as part of the gene, which included nucleotide modifications incorporated at their 5′ and 3′ ends as a result of primer design which were necessary to allow their cloning (or resulted from this process) in E. coli, Salmonella typhimurium, or other negative Gram negative or Gram positive bacteria.

24. The tbpB gene of the Chilean B:4:NT strain modified at its ends to obtain TbpB according to claim 19, wherein in its total or modified length, or considering just a part thereof, it is appropriate to be used in the design of primers for a PCR reaction, real time PCR or any other variant of PCR type amplification (such as RAPD, AFLP or the like) that can be used for diagnosis purposes in preventing or following-up meningitis caused by Neisseria meningitidis.
